(19) United States
(12) Patent Application Publication
(10) Pub. No.: US 2022/0355014 A1
(43) Pub. Date: Nov. 10, 2022

(54) METHOD AND APPARATUS FOR CONTROLLING ANTICOAGULATION DURING EXTRACORPOREAL BLOOD TREATMENT

(71) Applicant: B. Braun Avitum AG, Melsungen (DE)

(72) Inventor: Peter Mandry, Dresden (DE)

(21) Appl. No.: 17/760,529

(22) PCT Filed: Sep. 18, 2020
(86) PCT No.: PCT/EP2020/076133

§ 371 Date: Mar. 15, 2022

(30) Foreign Application Priority Data
Sep. 20, 2019 (DE) 10 2019 125 355.1

(51) Int. Cl. A61M 1/36; A61M 1/34

(52) U.S. Cl. CPC A61M 1/3672; A61M 1/342; A61M 1/3486; A61M 2205/3334; A61M 2202/0415; A61M 2230/207

(57) ABSTRACT

A method and device for controlling anticoagulation during blood treatment. The method includes conveying blood in a first line section, supplying biologically and/or pharmacologically active substances of negative total charge to the blood, separating the blood into corpuscular blood components and blood plasma, conveying the blood plasma in a second line section via an anion exchanger, bringing the blood plasma and corpuscular blood components together in a third line section, determining a first flow rate of blood plasma in the first line section, determining a second flow rate of blood plasma in the second line section, setting a quantity of biologically and/or pharmacologically active substances based on a ratio of the first and second flow rates such that, after the blood plasma and corpuscular blood components are brought together, a concentration of the biologically and/or pharmacologically active substances in the third line section meets a target value.

### CROSS-REFERENCES TO RELATED APPLICATIONS

This application is the United States national stage of International Application No. PCT/EP2020/076133, filed Sep. 18, 2020, and claims priority to German Application No. 10 2019 125 355.1, filed Sep. 20, 2019. The contents of International Application No. PCT/EP2020/076133 and German Application No. 10 2019 125 355.1 are incorporated by reference herein in their entireties.

### FIELD

The present invention relates to a method for controlling anticoagulation during extracorporeal blood treatment on an apparatus for extracorporeal blood treatment.

### BACKGROUND

A sepsis is one of the most serious complications of acute infections and infectious diseases and belongs to one of the most frequent and most cost intensive diseases in the inpatient sector. As a consequence, the sepsis represents a significant challenge for health systems all over the world. Cost factors which are associated with the treatment of sepsis patients are, inter alia, the frequent readmissions of patients to the hospital and the in many cases long-lasting effects of the disease, as for example long-term ventilation and the necessity of dialysis, which a sepsis disease often entails. But also the costs which are involved with an occurring long-term incapacity to work and an early retirement associated therewith cannot be ignored. In the USA, sepsis is listed at the top of the hospital treatment costs with annual costs of 24 billion U.S. Dollars. By comparison, the direct treatment costs of a sepsis in Germany in the outpatient and inpatient sector were estimated at 7.5 billion Euros in 2013.

The life-threatening condition of a sepsis comes about when the body's defense reactions can no longer restrict an infection—which is mostly caused by bacteria, but potentially also by viruses, fungi or parasites—and the consequences of said infection to a limited area. Consequently, an excessive defense reaction of the body occurs which leads to a damage of the own tissues and organs. If a sepsis will not be diagnosed and treated in good time, the patient's condition can deteriorate dramatically within a very short time and can lead to a multiple organ failure, for example of the heart, the lung, the liver and the kidneys, and/or to a septic circulatory shock and can end in death.

A key role in the excessive defense reaction of the body is played by the so-called lipopolysaccharides (LPS) which belong to the group of endotoxins. LPS are relatively thermostable compounds which consist of a hydrophilic polysaccharide fraction and a lipophile lipid fraction and which represent a main component of the outer cell wall of gram-negative bacteria, such as salmonellae, Escherichia coli or legionellae. Even intravenous doses of 1 ng/kg body weight per hour can cause inflammatory reactions in humans, which is why LPS are regarded as extremely toxic. LPS are released into the blood stream during the cell division of proliferating gram-negative bacteria or during the lysis/destruction of said bacteria by antibiotics or the human complement system. The human immune system recognizes these endotoxins and, as a consequence, initiates a plurality of defense reactions and inflammatory reactions which lead to an overproduction of regulatory proteins, so-called cytokines. Such a systematic cytokine overproduction can, in turn, lead to a damaging of blood vessels and to a septic shock, accompanied by a disseminated intravascular coagulation and a multiple organ failure.

Therefore the removal of LPS from the blood of affected patients represents a promising approach in order to rapidly contain the initiated defense and inflammatory reactions and to reduce the production of cytokines. As Falkenhagen et al. (Int J Artif Organs 2014; 37 (3): 222-232) have been able to demonstrate within the framework of the examination of the effectiveness of different commercially available endotoxin adsorbers, only adsorbers having surfaces modified with diethylaminoethyl cellulose (DEAE)—which, however, have not been regarded as clinically applicable up to now—enable an effective removal of LPS from blood serum. According to the carried-out study, such an adsorber is an anion exchanger which has the ability to bind molecules of negative charge. For this purpose, the anion exchanger has a cationic material, in this case via a DEAE-modified surface, which is covalently bonded to a solid, insoluble matrix, and neutralizing anions which are ionically bonded thereto and which are exchanged by the binding of other anions.

During a blood treatment in the course of an extracorporeal therapy so-called anticoagulants/coagulation inhibitors, as for example heparin, are added to the blood of the respective patient in an extracorporeal circuit in order to inhibit a blood coagulation of the blood to be treated. Thereby, the concentration of the anticoagulant is of particular importance in the blood treatment of sepsis patients or of patients with a septic shock by means of extracorporeal therapies, as a concentration that is too low will lead to a coagulation of the blood and, thus, to a therapy stop, whereas a concentration of the anticoagulant that is too high can, when the treated blood is guided back into the body of the patient, lead to internal bleedings and even to death. As a result of this, a constant concentration of the anticoagulant agent is essential for a successful extracorporeal treatment of the blood of sepsis patients.

Heparin is a preferred anticoagulant which is used in particular in the intensive-care medicine for the treatment of a sepsis. Heparins are variably esterified glycosaminoglycans having a multitude of negative charges that unfold their anticoagulant effect from a chain length of five monosaccharides and above. By a binding to the protease inhibitor antithrombin III, heparin has the effect that the binding of antithrombin III to coagulation factors takes place approximately a thousand times faster, and, hence, the blood coagulation is inhibited. Because of its negative total charge, heparin, however, is not suitable for the use in the course of an extracorporeal blood treatment for the removal of LPS at an anion exchanger which is coated with DEAE, as, apart from LPS, the anion exchanger will also bind heparin. A removal of heparin will, however, as described above, result in a coagulation of the blood of the patient and, hence, will lead to a stop of the therapy. Furthermore, sepsis patients are treated with low doses of heparin for the prevention of a thrombosis. And also this heparin would be removed by an anion exchanger which is coated with DEAE. Consequently, there is a need for therapy possibilities of sepsis patients or of patients with a septic shock which enable an effective removal of LPS without creating any side effects or risks for the patient.

From the state of the art there is known the so-called HELP (heparin induced extracorporeal LDL precipitation) method which, inter alia, is disclosed in DE 31 35 814 A1. So far, the HELP method has been used for the chronic treatment of patients with a congenital lipometabolic disorder, the severe primary hypercholesterolemia. In the course of this, the previously separated plasma is mixed with heparin and acetate buffer, whereby the pH value of the plasma decreases. The precipitate produced under these conditions and consisting of LDL, fibrinogen and heparin is subsequently filtered out and removed from the plasma circulation. The then still remaining excess precipitating agent heparin is selectively adsorbed in a further step at a DEAE cellulose anion exchanger and is removed. By the removal of the buffer solution by means of a dialysis and an ultrafiltration, in the following the physiological pH value is restored so that the treated plasma can again be mixed with the blood cells and eventually be returned to the patient.

In order to avoid the coagulation of the blood and to compensate for the removal of heparin by the anion exchanger, a larger bolus of heparin is added to the blood of the patient within the scope of the HELP method as disclosed in DE 31 35 814 A1. On grounds of the above mentioned excessive defense reaction of the body, the blood coagulation in sepsis patients or in patients with a septic shock is, however, disturbed and instable, for what reason a constant heparin concentration is essential for such patients. On that account, a simple overdosing of heparin, as for example in patients who are treated by means of the HELP method, is not possible in the case of sepsis patients or patients with a septic shock and is even life-threatening.

Also EP 0 705 845 B1 discloses a method which, to a large extent, proceeds analogously to the above described HELP method. In this method the simultaneous removal of tumor necrosis factor alpha (TNFα) and bacterial LPS from an aqueous liquid is described. To this end, also here a larger bolus of heparin is added to the blood or the plasma after removing corpuscular blood components if necessary, whereby a precipitation of TNFα is induced. After a filtration or centrifugation of the TNFα precipitate, the excess heparin is removed by a binding thereof to an anion exchanger modified with DEAE cellulose. Apart from heparin, however, due to its negative charge also LPS will be removed effectively by binding to the anion exchanger. Similar to the HELP method, also in the method as disclosed in EP 0 705 845 B1 the physiological pH value of the blood or plasma will be regenerated subsequently by an ultrafiltration and/or by an additional dialysis step.

The simulation of the method has shown that, without any subsequent dosing, 98% of the heparin will be eliminated from the plasma by binding to the anion exchanger. A removal of heparin to such an extent has, however, as was already indicated above, the disadvantage that its blood-thinning effect is minimized or disappears and that the blood coagulates. A heparin concentration that is too low leads, however, as described above, to a stop of the extracorporeal blood treatment, since a continuance of the therapy based thereon increases the risk that possible blood clots resulting from the coagulation are guided back to the patient and can possibly cause thrombi in the body of the patient which can lead to serious complications, as for example a heart attack or an apoplectic stroke. Conversely, a subsequent dosing of heparin that is too large can lead to internal bleedings. Hence, a sufficient safety of the patient is not given when the above method is used in practice.

The use of other anticoagulants in such methods is, on the other hand, often less well clinically tested or involves high costs, which is why they find less application in practice.

US 2016/0038666 A1 discloses systems and methods for performing a kidney replacement therapy having or using a dialyzer, control components, sorbent cartridge and fluid reservoirs which are configured to be of a weight and size suitable to be worn or carried by an individual requiring treatment. The system for performing a kidney replacement therapy has a controlled compliance dialysis circuit, where a control pump controls the bi-directional movement of fluid across a dialysis membrane. The dialysis circuit and an extracorporeal circuit for circulating blood are in fluid communication through the dialysis membrane. The flux of fluid moving between the extracorporeal circuit and the dialysis circuit is modified by the rate at which the control pump is operating such that a rate of ultrafiltration and convective clearance can be controlled. The system provides for the monitoring of the inlet and outlet conductivity of the sorbent cartridge to provide a facility to quantify or monitor the removal of urea by the sorbent cartridge.

Thus, US 2016/0038666 A1 relates to the removal of cations of positive total charge by means of a cation exchanger, which is directed at dialysis patients with a chronic kidney failure. Therein it is disclosed that, for the dialysis, for the purpose of diminishing the concentration of urea, in a first step urea has to be separated by means of the enzyme urease into positively charged ammonium ions (NH4)+ and carbon dioxide. In a step following said enzymatic splitting, the ammonium ions (NH4)+ are extracted from the blood by means of an ion exchanger stage, for which a cation exchanger has to be used.

AT 509 192 A4 discloses an alternative to the use of anion exchangers on the basis of the other principle of sorption, namely a sorbent for the removal of endotoxins from a biological fluid, comprising a water-insoluble, porous carrier with a neutral, hydrophobic surface. Thereby, the surface of the carrier has an adsorptive coating consisting of polymyxin and albumin, wherein polymyxin and albumin are noncovalently bonded to the surface of the carrier. Hence, AT 509 192 A4 deals with the immobilization of polymyxin, that is earlier described as neurotoxic and nephrotoxic, in the interests of patient safety.

### SUMMARY

Consequently, the object of the present invention is to provide a method and an apparatus for enabling an effective elimination of LPS by using an anion exchanger and the simultaneous use of a number of/at least one biologically and/or pharmacologically active substance(s) of negative charge, in particular of heparin as anticoagulant, within the context of an extracorporeal blood purification for the treatment of sepsis patients or of patients with a septic shock. In this connection, a further object of the invention is to control the adding of the number of biologically and/or pharmacologically active substances of negative charge, in particular of heparin, during the therapy in such a way that the concentration of the number of biologically and/or pharmacologically active substances of negative charge in the extracorporeal circuit remains constant despite the adsorption at the anion exchanger.

A further object of the present invention in this connection is also to increase the safety of sepsis patients or of patients with a septic shock during the extracorporeal blood purification by avoiding undesirable side effects caused by blood clots or bleedings and by avoiding an interruption of the therapy, and to generally improve the therapy of sepsis.

According to a first aspect of the disclosure, one method for controlling anticoagulation during extracorporeal blood treatment on an apparatus for extracorporeal blood treatment comprises the following steps of: conveying blood in a first line section, in particular by means of a blood pump; supplying heparin to the blood in the first line section, in particular by means of a heparin pump; separating the blood added with heparin into corpuscular blood components and blood plasma, in particular by means of a plasma separator; conveying the separated blood plasma, in particular by means of a plasma pump, in a second line section via an anion exchanger for adsorption of lipopolysaccharides; and bringing the treated blood plasma and the corpuscular blood components together in a third line section. In the course of this, the method is characterized by the steps of: determining a first volumetric flow rate of the blood plasma fraction in the first line section before the supplying of heparin, in particular at the blood pump; determining a second volumetric flow rate of the blood plasma in the second line section upstream or downstream of the anion exchanger, in particular at the plasma pump; and setting a quantity of the heparin which is supplied to the blood before the separating, on the basis of the ratio of the first volumetric flow rate and the second volumetric flow rate, in such a way that, after the treated blood plasma and the corpuscular blood components have been brought together, a concentration of heparin in the third line section obtains a predetermined, in particular constant, target value. As, due to its functional principle, apart from heparin the anion exchanger also removes other plasma proteins of negative total charge, as for example coagulation factors, with the aid of the mathematical model underlying the method, a number of/at least one biologically and/or pharmacologically active substance(s) of negative total charge can generally be supplied by means of the above method. In the following, heparin is used as a synonym for all said biologically and/or pharmacologically active substances.

Accordingly, the present disclosure comprises in general a method for controlling anticoagulation during extracorporeal blood treatment on an apparatus for extracorporeal blood treatment, said method comprising the steps of: conveying blood in a first line section, in particular by means of a blood pump; supplying a number of biologically and/or pharmacologically active substances of negative total charge, in particular heparin, to the blood in the first line section, in particular by means of a pump; separating the blood added with the number of biologically and/or pharmacologically active substances of negative total charge into corpuscular blood components and blood plasma, in particular by means of a plasma separator; conveying the separated blood plasma, in particular by means of a plasma pump, in a second line section via an anion exchanger for adsorption of lipopolysaccharides; and bringing the treated blood plasma and the corpuscular blood components together in a third line section. Thereby, the method is characterized by the steps of: determining a first volumetric flow rate of the blood plasma fraction in the first line section before the supplying of the number of biologically and/or pharmacologically active substances of negative total charge, in particular at the blood pump; determining a second volumetric flow rate of the blood plasma in the second line section upstream or downstream of the anion exchanger, in particular at the plasma pump; and setting a quantity of the number of biologically and/or pharmacologically active substances of negative total charge, which substances are supplied to the blood before the separating, on the basis of the ratio of the first volumetric flow rate and the second volumetric flow rate, in such a way that, after the purified blood plasma and the corpuscular blood components have been brought together, a concentration of the number of biologically and/or pharmacologically active substances of negative total charge in the third line section obtains a predetermined, in particular constant, target value.

In other words, according to the above described aspect, on an apparatus for extracorporeal blood treatment, blood drawn in by means of a blood pump will first of all be added with heparin which is supplied by means of a pump, in particular by means of a heparin pump, and subsequently will be separated into corpuscular components and blood plasma in a plasma separator. While the corpuscular components will stay in the plasma separator together with a fraction of the blood plasma, another fraction of the blood plasma will be guided via an anion exchanger where the LPS being present in the blood plasma will be adsorbed and, consequently, removed. As, apart from LPS, also heparin will be adsorbed at the anion exchanger because of its negative charge, such a quantity of heparin will be added to the blood by means of the heparin pump before the separating that, after the treated blood plasma and the corpuscular components have been brought together, the heparin concentration will obtain a predetermined constant target value. In order to guarantee the constancy of the heparin concentration, a quantity of heparin which is to be supplied by means of the heparin pump will be set in such a way that it is based on a volumetric flow rate of the blood plasma fraction at the blood pump before the supplying of the heparin and on a volumetric flow rate of the blood plasma at the plasma pump upstream or downstream of the anion exchanger. In this way, the use of an anion exchanger and the simultaneous use of heparin as an anticoagulant will become possible within the context of an extracorporeal blood treatment, in particular for the blood treatment of a sepsis.

Thereby, it shows up as an advantage of the disclosure that it can be managed in this way, i.e. by accepting or bypassing the lack of selectivity as a technical disadvantage or as an obstacle of fundamental nature to be overcome, that, on the one hand, the technical overall objective of a removal of highly toxic endotoxins and, on the other hand, an avoidance of a blood coagulation by means of an effective suppression of the blood coagulation cascade by using blood coagulation inhibitors, in particular heparin, can nevertheless be achieved by a constant target value concentration.

It is furthermore advantageous that the presently disclosed method or the corresponding apparatus enable a successful extracorporeal blood treatment already below a complete extraction of endotoxins. In this respect, already a partial removal of the endotoxins has positive effects during one passage for the blood purification.

According to one aspect of the disclosure, the method can further comprise the steps of: determining an actual value of the concentration of the number of biologically and/or pharmacologically active substances of negative total charge in the third line section; and controlling the concentration of the number of biologically and/or pharmacologically active substances of negative total charge in the third line section on the basis of the determined actual value, the ratio of the first volumetric flow rate and the second volumetric flow rate and the supplied quantity of the number of biologically and/or pharmacologically active substances of negative total charge in the first line section. Such a control enables a continuous automatic adjustment of the concentration of the number of biologically and/or pharmacologically active substances of negative total charge in the third line section by taking into account possible disturbance variables. Thus, also in this manner a constant concentration of the number of biologically and/or pharmacologically active substances of negative total charge, in particular of heparin, in the third line section is guaranteed, which is, as already explained above, of an essential importance for the blood treatment of sepsis patients or of patients with a septic shock.

In order to be able to determine the above described volumetric flow rate of the blood plasma fraction in the first line section before the supplying of the heparin, the method can furthermore comprise the following steps of: determining a hematocrit value of the blood in the first line section; determining a volumetric flow rate of the blood in the first line section, in particular determining a conveying rate of the blood pump; and determining the volumetric flow rate of the blood plasma fraction in the first line section on the basis of the determined volumetric flow rate of the blood in the first line section and the determined hematocrit value. By the determination of the hematocrit value which reflects the percentage of the cell components in the entire volume of the blood and is dependent on the patient, and the determination of the volumetric flow rate of the blood in the first line section or of the conveying rate of the blood pump, a direct conclusion can be drawn as to the volumetric flow rate of the blood plasma fraction in the first line section which, eventually, is required for the calculation of the heparin quantity to be supplied to the blood in the first line section.

According to a further aspect of the disclosure, the method can comprise the step of: setting the second volumetric flow rate of the blood plasma flowing through the anion exchanger, in particular by means of the plasma pump. This means that before the start of and/or during the blood treatment, the volumetric flow rate of the blood plasma flowing through the anion exchanger or the conveying rate of the plasma pump for conveying a preselected quantity of blood plasma can be adjusted by an operator, as for example a medical doctor or hospital staff, at the apparatus for extracorporeal blood treatment and in dependence on the desired therapy conditions. Thereby the time course of the extracorporeal blood treatment and the rate of fluid flow through the anion exchanger can be flexibly modified. Alternatively or additionally, the conveying rate of the blood pump can be set and adjusted at the apparatus.

As already described above, apart from LPS also heparin is adsorbed at the anion exchanger due to its negative total charge. Furthermore, however, also other plasma proteins or medicines or agents of negative total charge which are present in the plasma can bind to the anion exchanger and can, thus, be removed from the blood plasma. In order to compensate for an undesired removal of further plasma proteins and/or agents, according to another aspect of the disclosure the above described method can comprise the step of: additionally supplying medicines and/or blood-inherent substances, which are adsorbed by the anion exchanger, into the third line section.

For the corresponding implementation and correct execution of the above described method, according to a further aspect of the disclosure the present disclosure comprises an anticoagulation control device for the use during an extracorporeal blood treatment, said anticoagulation control device comprising a means, in particular a blood pump, for conveying blood in a first line section; a means, in particular a heparin pump, for supplying heparin to the blood in the first line section; a means, in particular a plasma separator, for separating the blood added with heparin into corpuscular blood components and blood plasma; a means, in particular a plasma pump, for conveying the separated blood plasma in a second line section via an anion exchanger for adsorption of lipopolysaccharides; and a means for bringing the treated blood plasma and the corpuscular blood components together in a third line section. Thereby, the anticoagulation control device is characterized by a means for determining a first volumetric flow rate of the blood plasma fraction in the first line section before the supplying of the heparin, in particular at the blood pump; a means for determining a second volumetric flow rate of the blood plasma in the second line section upstream or downstream of the anion exchanger, in particular at the plasma pump; and a control unit which is adapted to control a heparin quantity supplied to the blood before the separating, on the basis of the ratio of the first volumetric flow rate and the second volumetric flow rate, in such a way that, after the purified blood plasma and the corpuscular blood components have been brought together, a concentration of the heparin in the third line section obtains a predetermined, in particular constant, target value. Thereby, a means for determining the second volumetric flow rate can for example be the conveying rate of the pump or a sensor, as for example a flow sensor or a pressure sensor. Thereby, the anticoagulation control device is in general adapted in such a way that a number of biologically and/or pharmacologically active substances of negative total charge can be supplied in the same manner as heparin.

Accordingly, the present disclosure comprises in general an anticoagulation control device for the use during an extracorporeal blood treatment, the anticoagulation control device comprising: a means, in particular a blood pump, for conveying blood in a first line section; a means, in particular a pump, for supplying a number of biologically and/or pharmacologically active substances of negative total charge, in particular heparin, to the blood in the first line section; a means, in particular a plasma separator, for separating the blood added with the number of biologically and/or pharmacologically active substances of negative total charge into corpuscular blood components and blood plasma; a means, in particular a plasma pump, for conveying the separated blood plasma in a second line section via an anion exchanger for adsorption of lipopolysaccharides; and a means for bringing the treated blood plasma and the corpuscular blood components together in a third line section. Thereby, the anticoagulation control device is characterized by a means for determining a first volumetric flow rate of the blood plasma fraction in the first line section before the supplying of the number of biologically and/or pharmacologically active substances of negative total charge, in particular at the blood pump; a means for determining a second volumetric flow rate of the blood plasma in the second line section upstream or downstream of the anion exchanger, in particular at the plasma pump; and a control unit which is adapted to control a quantity of the number of biologically and/or pharmacologically active substances of negative total charge, which substances are supplied to the blood before the separating, on the basis of the ratio of the first volumetric flow rate and the second volumetric flow rate in such a way that, after the treated blood plasma and the corpuscular blood components have been brought together, a concentration of the number of biologically and/or pharmacologically active substances of negative total charge in the third line section obtains a predetermined, in particular constant, target value.

Thereby, the fundamental dilemma of a lack of selectivity of anion exchangers with regard to the substances or materials or protagonists or molecules, all equally of negative total charge, namely on the one hand of endotoxins, in particular the lipopolysaccharides, which have to be separated from the blood as highly toxic substances for a (sepsis) patient, versus on the other hand of coagulation inhibitors like, for example, heparin as a substance essential for the survival of the (sepsis) patient, is overcome by the proposed disclosure.

Furthermore, the anticoagulation control device can comprise a means for determining an actual value of the concentration of the number of biologically and/or pharmacologically active substances of negative total charge in the third line section, and a means for controlling the concentration of the number of biologically and/or pharmacologically active substances of negative total charge in the third line section on the basis of the determined actual value, the ratio of the first volumetric flow rate and the second volumetric flow rate, and the supplied quantity of the number of biologically and/or pharmacologically active substances of negative total charge in the first line section. Thereby, the means for controlling the concentration of the number of biologically and/or pharmacologically active substances of negative total charge in the third line section can be a concentration control means or a controller. Thereby, the concentration control means or the controller is in particular used for a, quasi dynamic or automatic, control of the heparin concentration as a target value/reference variable on the basis of a measured actual value of the heparin concentration as a control variable which is based on a, quasi one-time, target value setting or target value control of the heparin concentration. This serves as a control variable for the, in particular dynamic and/or in real-time monitored, monitoring of the actual heparin concentration in the blood which is guided back to the (sepsis) patient.

The anticoagulation control device can further comprise a means for inputting a hematocrit value of the blood in the first line section and a means for determining the volumetric flow rate of the blood plasma fraction in the first line section on the basis of the conveying rate of the blood pump and the input hematocrit value. Thereby, the previously determined hematocrit value of the respective patient can be input for example via a user interface provided on the anticoagulation control device. The same user interface can also act as a means for inputting the conveying rate of the plasma pump and of the blood pump, as a means for inputting a desired heparin concentration in the third line section, which eventually shall flow to the patient. As in the case of the plasma pump, the conveying rate of the blood pump can be determined by for example sensors, like flow sensors or pressure sensors.

Preferably, the anticoagulation control device can comprise a means for determining a hematocrit value of the blood in the first line section, a means for determining a volumetric flow rate of the blood in the first line section, in particular for determining a conveying rate of the blood pump, and a means for determining the volumetric flow rate of the blood plasma fraction in the first line section on the basis of the determined volumetric flow rate of the blood in the first line section and the determined hematocrit value.

Preferably, the anticoagulation control device can comprise a means for setting the second volumetric flow rate of the blood plasma flowing through the anion exchanger, in particular the plasma pump and/or at the plasma pump.

Preferably, the anticoagulation control device can comprise a means for additionally supplying medicines and/or blood-inherent substances, which are adsorbed by the anion exchanger, into the third line section.

According to a further aspect of the disclosure, the anticoagulation control device can be designed in such a way that the blood pump is arranged in the first line section upstream of the pump, in particular of the heparin pump, and of the plasma separator. Alternatively, however, it is also possible to arrange the blood pump at another position within the extracorporeal blood circuit. Furthermore, the plasma pump is arranged in the second line section downstream of the plasma separator, but upstream of the anion exchanger. Also here an alternative arrangement of the plasma pump at another position within the second line section is possible.

According to the disclosure, the anion exchanger can have a surface which has been modified with diethylaminoethyl cellulose (DEAE). This is insofar of advantage as comparable materials or coatings of the anion exchanger, as for example polymyxin B (PMB), will remove LPS in practice (Falkenhagen et al., Int J Artif Organs 2014; 37 (3): 222-232) far worse from the blood serum. Hence, an anion exchanger with a DEAE-modified surface represents the material suited best and the most efficient solution for the adsorption of LPS from blood.

### BRIEF DESCRIPTION OF THE DRAWING FIGURES

The present disclosure will be described in more detail in the following by means of a preferred embodiment and under reference to the attached drawings, wherein:

FIG. 1 shows a representation for the illustration of a system structure according to the present disclosure;

FIG. 2 shows a diagram for the illustration of the decrease of the heparin concentration in blood during a simulated treatment without the substitution of heparin; and

FIG. 3 shows a diagram for the illustration of the heparin concentration in the blood during a simulated treatment with a substitution of heparin according to the present disclosure.

### DETAILED DESCRIPTION

In the following there will be described the embodiment of the present disclosure on the basis of the corresponding figures. Identical or functionally equivalent features are provided with the same reference numerals in the individual figures.

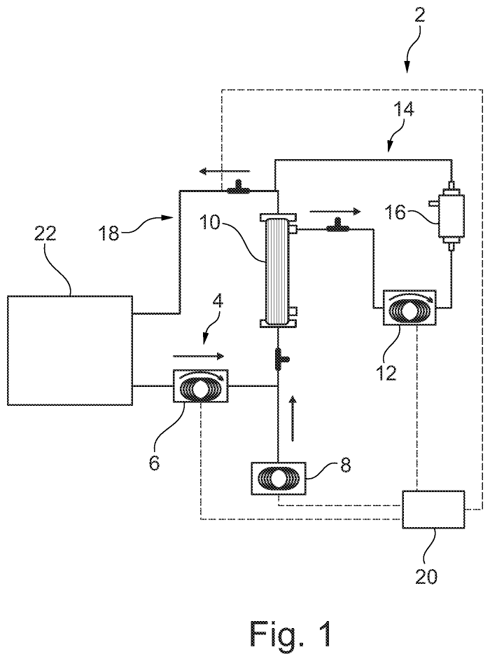

FIG. 1 shows a system structure of an anticoagulation control device 2 for the simulation of a method for an extracorporeal blood treatment. For the adequate implementation of the method, the anticoagulation control device 2 comprises a first line section 4 via which blood which shall be treated, in particular of a patient 22 undergoing the blood treatment, is drawn in by means of a, for example peristaltic, blood pump 6. Downstream of the blood pump 6 heparin will be supplied to the blood located in the first line section 4 by means of a pump 8, in particular a heparin pump 8, in order to guarantee that the blood will not coagulate. The blood added with heparin will finally be conveyed to a plasma separator 10 in which the blood will be separated into its corpuscular components, i.e. cellular components like erythrocytes or leucocytes, on the one hand and into blood plasma on the other hand. Thereby, the blood plasma contains ions and proteins, for example for blood coagulation. While the corpuscular components of the blood together with a fraction of the blood plasma will stay in the plasma separator 10, another fraction of the blood plasma will be sucked in via a vacuum produced by means of a plasma pump 12 into a second line section 14 and will be conveyed therein via an anion exchanger (16).

By the flowing through the anion exchanger 16, proteins of negative total charge will be bound/adsorbed on the surface of the anion exchanger 16 and will be removed thereby from the blood plasma. Thereby, a coating of the surface of the anion exchanger 16 with DEAE adsorbs above all LPS and heparin quite effectively. Due to the functional principle of the anion exchanger 16, apart from LPS and heparin, however, also medicines and other plasma proteins of negative total charge, as for example coagulation factors, will be removed. After the flowing through the anion exchanger 16, the treated blood plasma is guided into a third line section 18 which is located downstream of the plasma separator 10 and downstream of the anion exchanger 16, wherein in said third line section 18 the treated blood plasma will again be brought together with the corpuscular components which remained in the plasma separator 10 and with the one fraction of the blood plasma which remained untreated, and finally it will be conveyed via the third line section 18 back to the patient 22.

In order that the blood brought together after the flowing through the anion exchanger 16 will not coagulate due to the adsorption of the heparin and will thereby perhaps cause a stop of the blood treatment, the anticoagulation control device 2 is furthermore provided with a control unit 20 which controls the supplying of heparin in the first line section 4 by means of the heparin pump 8 in such a way that the heparin concentration in the third line section 18, which finally shall flow to the patient 22, obtains a predetermined, preferably constant, value. To this end, the control unit 20 detects a first volumetric flow rate of the blood plasma fraction in the first line section 4 determined at the blood pump 6 and a second volumetric flow rate of the blood plasma in the second line section 14 determined at the plasma pump 12 and brings them into relation. In order to determine the second volumetric flow rate, the anticoagulation control device 2 can for example be provided with sensors which are not shown in FIG. 1, said sensors detecting the volumetric flow rate at the plasma pump 12, or the anticoagulation control device 2 can derive it via the conveying rate of the plasma pump 12. In the same manner, in parallel to heparin also further biologically and/or pharmacologically active substances of negative total charge can be supplied and controlled.

For the determination of the volumetric flow rate of the blood plasma fraction in the first line section 4, apart from the detection of the volumetric flow rate of the blood at the blood pump 6 also the hematocrit value is required. Despite the fact that the hematocrit value is patient-dependent, it does not vary strongly in the course of the extracorporeal blood treatment of a patient so that, normally, it can be assumed to be constant. As in the case of the plasma pump 12, for the determination of the volumetric flow rate of the blood the anticoagulation control device 2 can comprise sensors or it can derive it by means of the conveying rate of the blood pump 6.

Furthermore, the anticoagulation control device 2 can comprise a means, for example in the form of a user interface, for inputting the hematocrit value of the blood in the first line section 4.

Furthermore and in analogy to the state of the art, the anticoagulation control device 2 can also comprise a dialysis unit and/or an ultrafiltration unit which are arranged in the third line section 18.

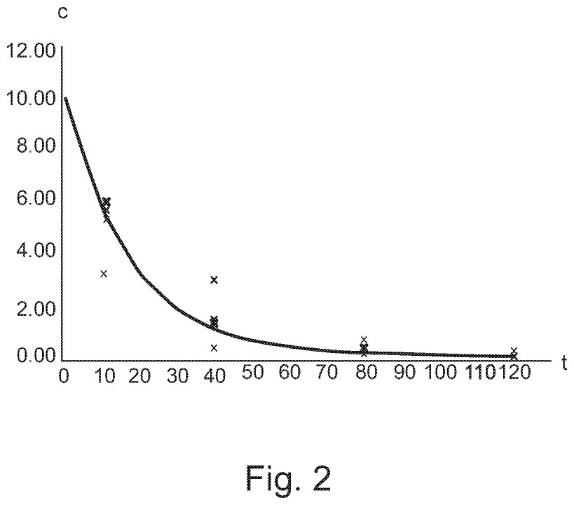

FIG. 2 shows a diagram in which the decrease of the heparin concentration in the blood of a patient 22 during a simulated treatment without any substitution of heparin is illustrated. Thereby, the heparin concentration c is represented in so-called International Units per milliliter [IU/ml] as a function of the treatment time tin minutes [min]. Before the treatment, i.e. at the point of time 0, the heparin starting concentration was set to 5 to 11 IU/ml at different conveying rates of the blood pump 6 and of the plasma pump 12, which is why four measurement values per measurement time were recorded. On the basis of FIG. 2 it becomes obvious that the heparin concentration in the blood decreases in an exponential rate with the progressing treatment. This means that the heparin concentration c in the blood sinks starting from the heparin starting concentration G in dependence on the treatment time t and a distance constant k. Said relationship can be represented as follows:

Δc=k**Gf(t))Δt (1)

If formula (1) is rearranged to the distance constant k and integrated over the treatment time t, the following relationship is obtained:

$$Δ c = k * ( G - f ( t ) ) Δ t \qquad ( 2 )$$

$$\frac{Δ c}{Δ t} = k * ( G - f ( t ) )$$

$$G = 0 \frac{Δ c}{\frac{Δ t}{f ( t )}} = k$$

$$ln \vert f ( t ) \vert = - k * t + b$$

$$f ( t ) - a * {a}^{- k * t}$$

$${c}_{eff} = {c}_{0} * {e}^{( - VSplasma / GVplasma ) * a}$$

wherein c_{eff} represents the effective heparin concentration at the point of time t, c₀ represents the heparin staring concentration in the blood, VSplasma represents the volumetric flow rate of the blood plasma in the second line section 14 at the plasma pump 12, and GVplasma represents the entire volume of the blood plasma fraction in the first line section 4, in particular at the blood pump 6, before the supplying of heparin by the heparin pump 8. From formula (2) there results in general the relationship that, for a constancy of the heparin concentration in the third line section 18, the heparin quantity to be supplied via the heparin pump 8 has to be increased such that the partial heparin flow via the anion exchanger 16 in the second line section 14 will be compensated for, wherein said partial heparin flow correlates with the plasma flow which can be set via the plasma pump.

In the clinical practice, however, no heparin concentration will be prescribed, but a heparin infusion with a defined volumetric flow rate. According to the present disclosure, a desired heparin volumetric flow rate HEP2 in the third line section 18, which finally shall flow to the patient 22, can be set before the treatment so that by including said parameter and on the basis of formula (2) the following relationship will be obtained:

HEP2=PF/(BF*(1−HK/100))*HEP1 (3)

where PF represents the plasma flow in the second line section 14, BF represents the blood flow in the first line section 4, HK represents the predetermined hematocrit value, and HEP1 represents the heparin quantity to be supplied via the heparin pump 8, which heparin quantity will be controlled by means of the control unit 20.

As already mentioned above, the plasma flow in the second line section 14 PF can be detected by the rate of rotation of the plasma pump 12 or for example by sensors, just as the blood flow in the first line section 4 BF which results from the determined conveying rate of the blood in the first line section 4 at the blood pump 6. Rearranged after HEP 1, for formula (3) the following is obtained:

HEP1=HEP2/(1−(PF/(BF*(1−HK/100))) (4)

In order to eventually calculate the heparin quantity to be supplied in the first line section 4 or the conveying rate of the heparin pump 8 by means of formula (4), an operator of the anticoagulation control device 2 inputs for example via the user interface HEP2, PF, BF and the determined HK of the patient 22 beforehand or also during the treatment. By means of the treatment data, the anticoagulation control device 2 can then automatically control the heparin quantity to be supplied in the first line section 4 or the conveying rate of the heparin pump 8.

In order to keep HEP2 constant even under the influence of possible interfering factors, the control unit 20 of the anticoagulation control device 2 can also be designed as a control unit. The control unit then controls HEP2 on the basis of an actual value determined in the third line section 18, the ratio of the volumetric flow rate of the blood plasma fraction in the first line section 4 before the supplying of the heparin and the volumetric flow rate of the blood plasma in the second line section 14 at the plasma pump 12.

Thus, one idea of the present disclosure is that, when from the permeate, i.e. the (partial) blood plasma volumetric flow in the second line section 14, which has been withdrawn from the plasma separator 10 by means of the plasma pump 12, the highly toxic LPS can only be extracted in the anion exchanger 16 by a simultaneous co-separation of heparin that likewise has a negative total charge, said loss quantity of heparin has to be substituted in the sense of a constant target concentration as required for example for a sepsis patient 22. Insofar as it can sometimes be a life-essential, detoxifying measure, it can be technically readily accepted that a first fraction of LPS cannot be extracted from the blood plasma without a second fraction of heparin. This also means that the anion exchanger 16 can wear out capacitively much faster as it would be the case in an ideal situation of an available separation matrix being selective specifically for the first fraction of LPS. In other words, the absolute aim of a life-saving detoxification is achieved when it is possible to use an excess of matrix of the anion exchanger 16 in a quasi <<uneconomic >> fashion.

In a further advantageous manner, the substitution via the heparin quantity supplied in the first line section by means of the heparin pump 8 serves to avoid a coagulation over all three line sections. Also in this point the present disclosure shows a further change of paradigm in that it departs from the teaching of the experts as described in the introduction to preferably not expose or only slightly expose an anion exchanger to heparin.

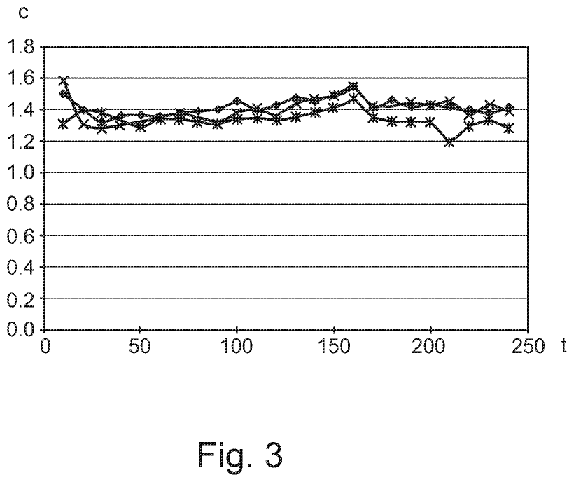

In contrast to FIG. 2, FIG. 3 shows a diagram for the illustration of the heparin concentration in the blood of a patient 22 during a simulated treatment with a substitution of heparin according to the present disclosure. Also in FIG. 3 the heparin concentration c is represented in so-called International Units per milliliter [IU/ml] as a function of the treatment time tin minutes [min]. In this case a heparin concentration of 1.4 IU/ml was previously set which finally shall be present in the third line section 18 and shall be supplied to the patient 22. The control unit 20 controls the heparin quantity to be supplied in the first line section 4 or the conveying rate of the heparin pump 8 in FIG. 3 on the basis of formula (4). In this way, HEP2 can be kept constant over the entire treatment duration.

Thus, the present disclosure enables the use of anion exchangers 16 and the simultaneous use of heparin in the course of an extracorporeal therapy. Thereby the material suited best for the removal of endotoxins from the blood according to Falkenhagen et al. (Int J Artif Organs 2014; 37 (3): 222-232) can be applied in an extracorporeal therapy in humans in case of a sepsis.

1. A method for controlling anticoagulation during extracorporeal blood treatment on an apparatus for extracorporeal blood treatment, said method comprising the steps of:
conveying blood in a first line section;
supplying at least one biologically and/or pharmacologically active substance of negative total charge to the blood;
separating the blood into corpuscular blood components and blood plasma;
conveying the blood plasma in a second line section via an anion exchanger for adsorption of lipopolysaccharides;
bringing the blood plasma and the corpuscular blood components together in a third line section;
determining a first volumetric flow rate of the blood plasma in the first line section before supplying the at least one biologically and/or pharmacologically active substance of negative total charge to the blood;
determining a second volumetric flow rate of the blood plasma in the second line section upstream or downstream of the anion exchanger; and
setting a quantity of the at least one biologically and/or pharmacologically active substance of negative total charge based on a ratio of the first volumetric flow rate and the second volumetric flow rate, in such a way that, after the blood plasma and the corpuscular blood components are brought together, a concentration of the at least one biologically and/or pharmacologically active substance of negative total charge in the third line section meets a predetermined target value.

2. The method according to claim 1, further comprising the steps of:
determining an actual value of the concentration of the at least one biologically and/or pharmacologically active substance of negative total charge in the third line section; and
controlling the concentration of the at least one biologically and/or pharmacologically active substance of negative total charge in the third line section based on the actual value, the ratio of the first volumetric flow rate and the second volumetric flow rate, and the quantity of the at least one biologically and/or pharmacologically active substance of negative total charge.

3. The method according to claim 1, further comprising the steps of:
determining a hematocrit value of the blood in the first line section;
determining a volumetric flow rate of the blood in the first line section; and
determining the volumetric flow rate of the blood plasma fraction in the first line section on the basis of the determined volumetric flow rate of the blood in the first line section and the determined hematocrit value.

4. The method according to claim 1, further comprising the step of:
setting the second volumetric flow rate of the blood plasma flowing through the anion exchanger.

5. The method according to claim 1, further comprising the step of:
additionally supplying medicines and/or blood-inherent substances, which are adsorbed by the anion exchanger, into the third line section.

6. An anticoagulation control device for application during extracorporeal blood treatment, said anticoagulation control device comprising:
a blood pump for conveying blood in a first line section;
a pump for supplying at least one biologically and/or pharmacologically active substance of negative total charge to the blood in the first line section;
a plasma separator for separating blood added with the at least one biologically and/or pharmacologically active substance of negative total charge into corpuscular blood components and blood plasma;
a plasma pump, for conveying the blood plasma in a second line section via an anion exchanger for adsorption of lipopolysaccharides;
a means for bringing the blood plasma and the corpuscular blood components together in a third line section;
a means for determining a first volumetric flow rate of blood plasma in the first line section before the supplying of the at least one biologically and/or pharmacologically active substance of negative total charge; and
a means for determining a second volumetric flow rate of blood plasma in the second line section upstream or downstream of the anion exchanger;
wherein
a control unit which is adapted to control a quantity of the at least one biologically and/or pharmacologically active substance of negative total charge, which is supplied to the blood before the separating, based on a ratio of the first volumetric flow rate and the second volumetric flow rate, in such a way that, after the blood plasma and the corpuscular blood components are brought together, a concentration of the at least one biologically and/or pharmacologically active substance of negative total charge in the third line section meets a predetermined target value.

7. The anticoagulation control device according to claim 6, wherein:
a means for determining an actual value of the concentration of the at least one biologically and/or pharmacologically active substance of negative total charge in the third line section; and
a means for controlling the concentration of the at least one biologically and/or pharmacologically active substance of negative total charge in the third line section on the basis of the determined actual value, the ratio of the first volumetric flow rate and the second volumetric flow rate, and the supplied quantity of the at least one biologically and/or pharmacologically active substance of negative total charge in the first line section.

8. The anticoagulation control device according to claim 6, further comprising:
a means for inputting a hematocrit value of the blood in the first line section; and
a means for determining a volumetric flow rate of the blood plasma in the first line section based on a conveying rate of the blood pump and the hematocrit value.

9. The anticoagulation control device according to claim 6, further comprising:
a means for determining a hematocrit value of the blood in the first line section;
a means for determining a volumetric flow rate of the blood in the first line section; and
a means for determining the volumetric flow rate of the blood plasma in the first line section based on the volumetric flow rate of the blood in the first line section and the hematocrit value.

10. The anticoagulation control device according to claim 6, further comprising:
a means for setting the second volumetric flow rate of blood plasma.

11. The anticoagulation control device according to claim 6, further comprising:
a means for additionally supplying medicines and/or blood-inherent substances, which are adsorbed by the anion exchanger, into the third line section.

12. The anticoagulation control device according to claim 6, wherein the blood pump is arranged in the first line section upstream of the pump and of the plasma separator.

13. The anticoagulation control device according to claim 6, wherein the plasma pump is arranged in the second line section downstream of the plasma separator, but upstream of the anion exchanger.

14. The anticoagulation control device according to claim 6, wherein the anion exchanger has a surface modified with diethylaminoethyl cellulose.

15. The anticoagulation control device according to claim 6, wherein the predetermined target value is constant.

16. The method according to claim 1, wherein the predetermined target value is constant.
